(19) United States
(12) Patent Application Publication
(10) Pub. No.: US 2010/0292290 A1
(43) Pub. Date: Nov. 18, 2010

(54) NOVEL PROCESS TO PREPARE ALMOTRIPTAN

(76) Inventors: Vinayak Gore, Maharashtra (IN); Mahesh Gadkar, Herfordshire (GB); Kiran Pokharkar, Maharashtra (IN)

Correspondence Address: FULBRIGHT & JAWORSKI L.L.P.;Attn: MN IP Docket, 600 Congress Avenue, Suite 2400, Austin, TX, 78701, US

(21) Appl. No.: 12/671,095

(22) PCT Filed: Aug. 1, 2008
(86) PCT No.: PCT/GB08/50653

§ 371 Date: Jun. 1, 2010

(30) Foreign Application Priority Data
Aug. 2, 2007 (IN) 1492/MUM/2007

(51) Int. Cl. A61K 31/403; C07D 403/12; C07D 207/48; A61P 25/06

(52) U.S. Cl. 514/414; 548/467; 548/542

(57) ABSTRACT

The present invention relates to a novel process for the preparation of almotriptan and pharmaceutically acceptable salts thereof, which affords product conveniently and efficiently with commercially acceptable yields and purity. The present invention also relates to a novel synthetic intermediate used in the process.

### CROSS-REFERENCE TO RELATED APPLICATION(S)

This application is a Section 371 National Stage Application of International No. PCT/GB2008/050653, filed 1 Aug. 2008 and published as WO 2009/016414 A1 on 5 Feb. 2009, which claims priority from the India Application 1492/MUM/2007, filed 2 Aug. 2007, the contents of which are incorporated herein in their entirety for all purposes.

### FIELD OF THE INVENTION

The present invention relates to a novel process for the preparation of almotriptan and pharmaceutically acceptable salts thereof, which affords product conveniently and efficiently with commercially acceptable yields and purity. The present invention also relates to a novel synthetic intermediate used in the process.

### BACKGROUND OF THE INVENTION

Almotriptan, chemically named 3-[2-(dimethylamino)ethyl]-5-(pyrrolidin-1-ylsulfonyl-methyl)-1H-indole (I) is currently marketed, as the malate salt (II), for the treatment of the acute headache phase of migraine attacks with or without aura.

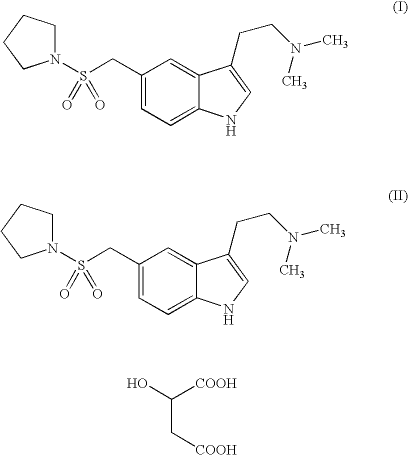

Almotriptan is structurally derived from tryptamine and is a medicine used to treat vascular headaches such as migraine. Almotriptan is a selective 5-hydroxytriptamine-1B/1D (5-HT1B/1D) receptor agonist, which belongs to the serotonin receptor agonist class of compounds. They are believed to work by causing vasoconstriction of arteries and veins that supply blood to the head.

Various patents describe processes for the preparation of almotriptan base, which can be converted into desired pharmaceutically acceptable salts.

The process for obtaining almotriptan base and pharmaceutically acceptable salts thereof disclosed in U.S. Pat. No. 5,565,447 is shown in Scheme 1. U.S. Pat. No. 5,565,447 describes the preparation 3,5-disubstituted indole derivatives such as almotriptan by decarboxylation of the intermediate 1-[[2-carboxy-3-(dimethylaminoethyl)-5-indolyl]methanesulfonyl]pyrrolidine using a copper oxide catalyst and quinoline as solvent. The above intermediate was prepared in four steps by following a process already reported in the literature, which affects the overall yield of the product. The process conditions reported for the decarboxylation require a very high temperature (190° C.) which in turn affects the quality of the almotriptan. Moreover, such a high temperature is difficult to achieve on a commercial scale. Also, it is difficult to separate almotriptan from quinoline, which is used as the solvent, due to their similar chemical characteristics. A multi-step work-up is needed to isolate the product and further chromatographic purification is essential to achieve the desired quality of the final compound.

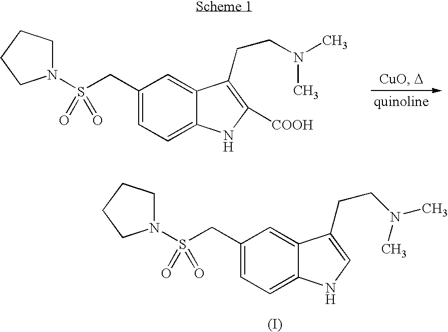

ES 2,084,560 describes a process for the preparation of almotriptan based on a Fischer indole synthesis using a phenyl hydrazine and 4-chloro-butyraldehyde diethyl acetal to afford 1-[[3-(2-aminoethyl)-5-indolyl]methanesulfonyl]pyrrolidine (see Scheme 2). The 1-[[3-(2-aminoethyl)-5-indolyl]methanesulfonyl]pyrrolidine formed was further treated with an 18% solution of formaldehyde and then sodium borohydride. After completion of the reaction and usual work-up of the reaction mass, 1-[[3-(2-dimethylaminoethyl)-5-indolyl]methanesulfonyl]pyrrolidine was obtained and subsequently converted into the marketed DL-malate salt. However, this process provides a poor yield of only about 20%. Furthermore, the process conditions give rise to the formation of polymeric impurities in substantial quantities, making the isolation of almotriptan very laborious and low yielding. The almotriptan obtained is not of adequate quality and needs further purification. It was observed that the HPLC purity was approximately 80-85% which is substantially lower than required for a pharmaceutical product. Expensive purification steps (chemical purification—acid base purification) and organic washings at various pHs have to be employed to achieve a reasonable purity (90-95%). The work-up procedure for the removal of degraded material formed in the cyclization reaction is very tedious and involves extraction with organic solvent to remove polar and non-basic impurities, in situ purification of crude almotriptan by preparation of an acid addition salt, and further purification using activated carbon before conversion into pharmaceutically acceptable salts. In addition, further processing to form the DL-malate salt is required to achieve a purity of more than 98.5%.

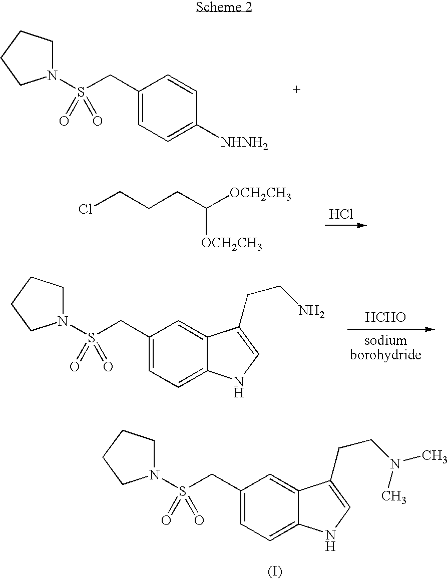

Another process for the preparation of almotriptan is described in WO 2006/129190 and illustrated in Scheme 3. 4-(1-Pyrrolidinylsulfonylmethyl)aniline was halogenated at the 2-position to obtain 2-iodo-4-(1-pyrrolidinylsulfonylmethyl)aniline. The 2-iodo-4-(1-pyrrodinylsulfonylmethyl)aniline was further coupled with 1-triethylsilyloxy-4-triethylsilyl-3-butyne by palladium catalyzed Heck coupling to obtain 5-(1-pyrrolidinyl-sulfonylmethyl)-1H-indole-3-ethanol. The 5-(1-pyrrolidinyl-sulfonylmethyl)-1H-indole-3-ethanol was converted into almotriptan succinate by using methanesulfonyl chloride, dimethylamine and succinic acid. The almotriptan succinate obtained was converted into almotriptan base which was then converted into almotriptan DL-malate. However, the preparation of 1-triethylsilyloxy-4-triethylsilyl-3-butyne involves the use of n-butyl lithium, which is neither convenient nor safe for a commercial scale production. Moreover, use of n-butyl lithium requires stringent reaction conditions, i.e. strict control on moisture content during the reaction, and special storage conditions leading to high manufacturing costs.

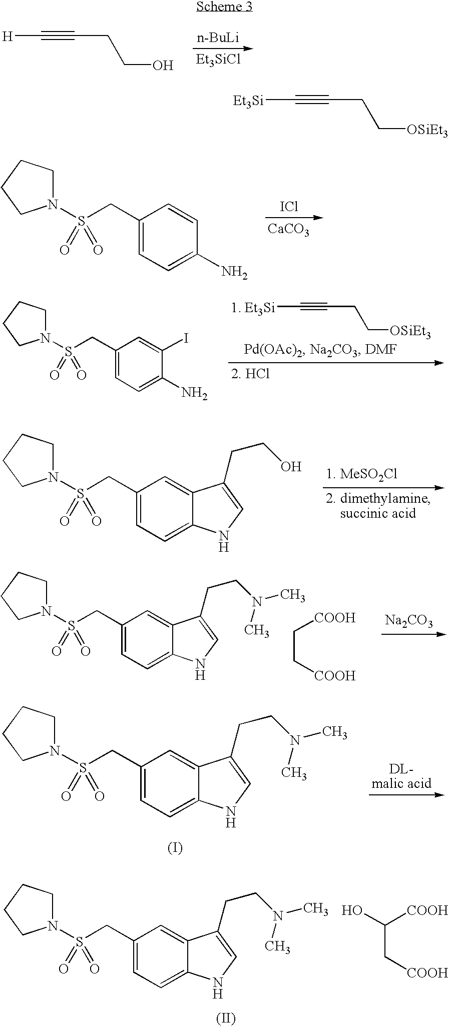

The synthetic scheme reported in Tetrahedron, 2001, vol. 57, pages 1041-1048 involves preparation of the indole ring via Heck cyclization. The sequential process involves multiple steps as shown in Scheme 4. 1-(4-Amino-benzenemethanesulfonyl)pyrrolidine was treated with bromine followed by trifluoroacetic acid for introduction of a bromo moiety at the 2-position and protection of the aniline nitrogen. Further, allylation was carried out using LDA and methyl 4-bromocrotonate. Heck cyclization was achieved using Pd(OAc)₂. The indole-3-acetic ester obtained was hydrolyzed into the corresponding acid, which was converted into the acid chloride and further to the dimethyl amide by reaction with dimethylamine in basic medium. Finally, reduction of the amide carbonyl gave the desired compound. However, this synthesis offers a very poor overall yield of almotriptan (less than 5%).

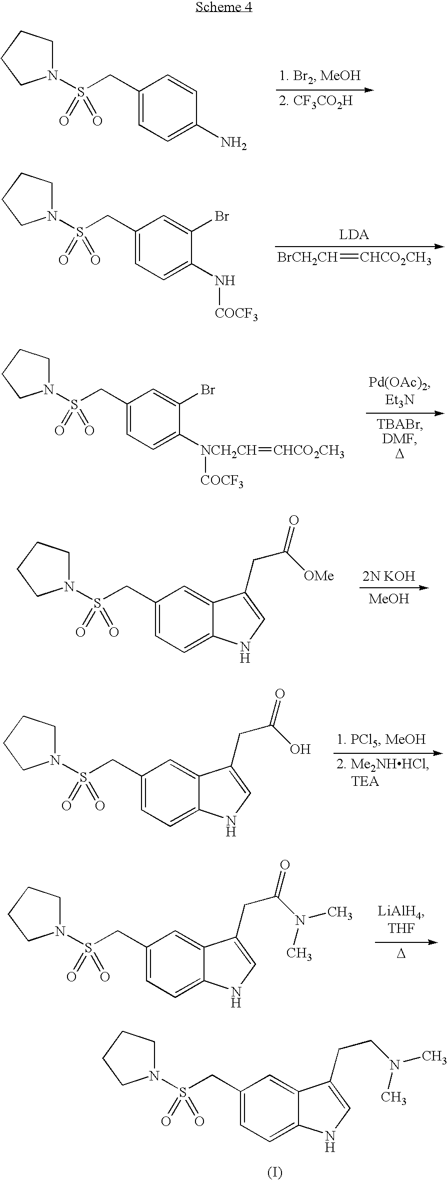

Surprisingly, the prior art does not report any process of making almotriptan via the shortest possible route of building up the indole nucleus from the corresponding amine, hydrazine or hydrazone and N,N-dimethylamino-butyraldehyde or a protected form thereof (e.g. an acetal). In all the reported syntheses, the required side chain was built in a sequential manner either from 2-acetyl-5-(dimethylamino)-pentanoic acid ethyl ester, 4-chloro-butyraldehyde diethyl acetal, 3-butyn-1-ol and methyl 4-bromocrotonate etc. The Fischer indole approach to obtain triptans such as zolmitriptan, rizatriptan, using N,N-dimethylamino-butyraldehyde is reported to be without significant degradation of the title molecules. However, it was observed that in the case of triptans having a sulfonamide functionality at C-5 (e.g. sumatriptan and almotriptan), similar Fischer indole conditions (e.g. indole formation at 85-90° C. for 3 to 8 hours) leads to significant degradation of the title molecules and hence there was a need to develop improved methods of synthesis for these molecules.

Consequently, all the processes disclosed in the prior art suffer from the disadvantages discussed above, such as multi-step synthesis, significant formation of degradation products, moderate to low yields and/or inappropriate reagents for commercial production.

Therefore, there is a need for a novel convenient process for the synthesis of almotriptan and pharmaceutically acceptable salts thereof, which provides the product conveniently with commercially acceptable yield and purity.

### SUMMARY OF THE INVENTION

The present inventors have very surprisingly found that in spite of having a sulfonamide functionality at C-5, it was possible to prepare almotriptan by using N,N-dimethylamino-butyraldehyde in the Fischer indole approach by a simple, convenient method which can be adapted as a “one-pot” process if required.

The invention relates to the design of optimum conditions for indole formation using N,N-dimethylamino-butyraldehyde or a protected form, such as the dimethyl acetal, under which almotriptan is stable and does not degrade. Preferably this is achieved by creating the right dilution, pH and temperature of the reaction medium and, if these conditions are followed, the process will be invariant of scale.

The present invention also provides a process for the preparation of almotriptan with novel work-up conditions to remove whatever degradants are formed during the formation of almotriptan to achieve the required quality and control on impurities and to achieve an impurity profile as per the ICH guidelines.

The present invention further provides quality almotriptan, as required by the ICH guidelines, alternatively by elution over an adsorbent using a mixture of solvents of defined composition, preferably mixed with an organic amine such as triethylamine.

In addition, the current invention offers a simple work-up procedure with optimum conditions for improved yield and quality with minimum contamination with process impurities. The process can be easily adopted on commercial scale as an efficient and convenient process.

A first aspect of the invention provides a process for the preparation of almotriptan or a pharmaceutically acceptable salt thereof, comprising:

(a) condensation of 1-(4-hydrazino-benzenemethanesulfonyepyrrolidine, or a pharmaceutically acceptable salt thereof, with N,N-dimethylamino-butyraldehyde, or a protected form thereof, to form hydrazone intermediate (V), or a protected form thereof,

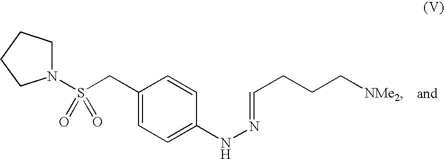

(b) cyclization of the hydrazone intermediate (V) to afford almotriptan.

Preferably the 1-(4-hydrazino-benzenemethanesulfonyl)pyrrolidine, or the pharmaceutically acceptable salt thereof, used in step (a) is prepared by diazotization of 1-(4-amino-benzenemethanesulfonyl)pyrrolidine, or a pharmaceutically acceptable salt thereof, followed by reduction. Preferably, the reduction of the diazo-compound is carried out using stannous chloride, sodium dithionite and sodium sulfite, but is preferably carried out using sodium sulfite.

The process according to the current invention is preferably a “one pot” process but alternatively, the hydrazone intermediate (V), or a protected form thereof, can be isolated if required.

The pharmaceutical salt of the intermediate amines or hydrazines used is preferably the hydrochloride salt. The N,N-dimethylamino-butyraldehyde is preferably used in the form of an acetal, such as a diacetal, such as the dimethyl acetal or diethyl acetal, preferably the dimethyl acetal.

Preferably, the condensation in step (a) is carried out at pH 0-3, most preferably at approximately pH 2.

Preferably the cyclization in step (b) is carried out at acidic pH, more preferably at pH 0-3, and most preferably at approximately pH 2.

Preferably, the cyclization in step (b) is carried out at 40-70° C., most preferably at 55-65° C.

In addition, the cyclization in step (b) is preferably carried out at high dilution such as 10-100 volumes dilution and typically at about 40 volumes dilution.

For the purposes of the present invention, “volumes dilution” means the quantity of solvent used relative to the starting material. For example, if 25 g of 1-(4-amino-benzenemethanesulfonyl)pyrrolidine are used as starting material and the reaction is carried out at 4 volumes dilution, this means that 25×4=100 ml solvent are used.

Preferably, the cyclization in step (b) is carried out in the presence of one or more mineral acids or Lewis acids, preferably selected from hydrochloric acid, sulfuric acid, acetic acid, phosphoric acid, trifluoroacetic acid or boron trifluoride. Preferably, the cyclization in step (b) is carried out in the presence of a suitable metal catalyst, such as palladium (II) acetate, palladium (II) chloride, Pd(P(C6H5)3)4, tris(dibenzylideneacetone)dipalladium (0) [Pd2(dba)3], zinc chloride or ruthenium complexes. Palladium (II) acetate is preferably used.

A preferred process of the invention involves isolating the almotriptan formed by extraction using one or more organic solvents, such as methyl acetate, ethyl acetate, isopropyl acetate, dichloromethane, chloroform, diethyl ether, tertiary butyl methyl ether, diisopropyl ether or mixtures thereof.

A particularly preferred process according to the invention is when almotriptan base is isolated using an adsorbent and an elution system. Preferably the adsorbent is selected from silica gel or different types of alumina, such as basic alumina or neutral alumina. Preferably the elution system is selected from a mixture of a solvent and an organic base, such as a mixture of an alcohol, acetate or chlorinated solvent and an organic amine, such as triethylamine, diethylamine, diisopropylamino, N-ethylisopropylamine, N,N-ethyldiisopropylamine, pyridine, pyrrolidone or a mixture thereof.

The process according to the first aspect of the invention can be used for the preparation of almotriptan base or a pharmaceutically acceptable salt of almotriptan, such as almotriptan malate.

Preferably the almotriptan or the pharmaceutically acceptable salt thereof obtained by the process according to the first aspect of the invention has a chemical purity of 96% or more, preferably 98% or more, preferably 99% or more, preferably 99.5% or more, preferably 99.85% or more (as measured by HPLC).

Preferably the almotriptan or the pharmaceutically acceptable salt thereof is obtained in a yield of 20% or more, preferably 25% or more, preferably 30% or more, preferably 35% or more, from 1-(4-hydrazino-benzenemethanesulfonyl)pyrrolidine or a pharmaceutically acceptable salt thereof.

Preferably the almotriptan or the pharmaceutically acceptable salt thereof is obtained on an industrial scale, preferably in batches of 50 g, 100 g, 500 g, 1 kg, 5 kg, 10 kg, 50 kg, 100 kg or more.

A second aspect of the invention is almotriptan or almotriptan malate as prepared by the process of the first aspect of the invention.

A third aspect of the invention is a pharmaceutical composition comprising almotriptan malate prepared according to the process of the first aspect of the invention.

A fourth aspect the invention is the use of almotriptan malate, as prepared by the process of the first aspect of the invention, in the preparation of a medicament for the treatment or prevention of migraine.

A fifth aspect of the invention is a method of treating or preventing migraine, comprising administering a therapeutically or prophylactically effective amount of almotriptan malate, as prepared by the process of the first aspect of the invention, to a patient in need thereof. Preferably the patient is a human.

A sixth aspect of the invention is the novel intermediate, hydrazone (V), or a protected form thereof:

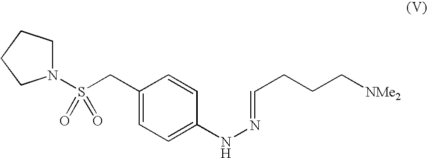

A seventh aspect of the invention is a process for the preparation of almotriptan, or a pharmaceutically acceptable salt thereof, preferably almotriptan malate, wherein the process utilizes the hydrazone intermediate (V), or a protected form thereof.

### DETAILED DESCRIPTION OF THE INVENTION

The present invention provides a novel convenient synthetic process for the synthesis of almotriptan and pharmaceutically accepted salts thereof by preferably using N,N-dimethylamino-butyraldehyde dimethyl acetal as outlined below in Schemes 5 to 8.

A “one pot” synthesis of almotriptan from 1-(4-amino-benzenemethanesulfonyl)pyrrolidine hydrochloride (III) is outlined in Scheme 5.

Diazotization of 1-(4-amino-benzenemethanesulfonyl)pyrrolidine hydrochloride (III) was carried out by using sodium nitrite (1.5 eq.) in the presence of hydrochloric acid at low temperatures (−10 to 5° C.). It is necessary to continue the reaction at lower temperature up to 8 hours to achieve complete conversion of 1-(4-amino-benzenemethanesulfonyl)pyrrolidine hydrochloride (III) into the corresponding diazonium hydrochloride salt. It was observed that if the reaction was terminated before 4-9 hours, unreacted 1-(4-amino-benzenemethanesulfonyl)pyrrolidine hydrochloride (III) was found as major impurity in the subsequent stage.

Reduction of the diazonium intermediate was carried out by using different reducing agents, such as stannous chloride, sodium dithionite and sodium sulfite. The best results were obtained when the reduction was carried out with sodium sulfite. Sodium sulfite (6 eq.) was dissolved in water (10-20 vol.) at 25-30° C. to obtain a clear solution. The diazonium salt solution obtained was added to the clear solution of sodium sulfite at 0-5° C. to avoid decomposition of the diazonium salt. After completion of the addition of the diazonium salt solution, the reaction mixture was stirred at 25-30° C. for 13-18 hours to achieve complete conversion of the diazonium salt to 1-(4-hydrazino-benzene-methanesulfonyl)pyrrolidine (IV). 1-(4-Hydrazino-benzenemethanesulfonyl)pyrrolidine (IV) was subsequently condensed with N,N-dimethylamino-butyraldehyde dimethyl acetal to afford the hydrazone intermediate (V).

The solution of 1-(4-hydrazino-benzenemethanesulfonyl)pyrrolidine (IV) was diluted up to 50 volumes with water and, after addition of N,N-dimethylamino-butyraldehyde dimethyl acetal at 25-30° C., the pH of the reaction mixture was adjusted with dilute HCl to pH 2. It was observed that the pH of the reaction mixture for this particular step was important to minimize degradation. The reaction mixture was further stirred at pH 2 at 25-30° C. for 5-6 hours for complete hydrazone formation.

Another important aspect of the present invention is the cyclization of hydrazone intermediate (V) to almotriptan base (I) as a “one pot” process.

Thus the pale yellow clear reaction mixture of hydrazone (V) was further subjected to heating at 55-65° C. for up to 10-12 hours for complete cyclization of the hydrazone intermediate (V) into almotriptan free base. It was observed that reaction parameters temperature (55-65° C.) and time (10-12 hours) were important for this reaction step to achieve complete and clean conversion. These reaction parameters also minimized the formation of degradation products. It is reported in the literature that sumatriptan, which also has a sulfonamide functional group, degrades under Fischer indole cyclization conditions.

After heating at 55-65° C. for 10-12 hours, the reaction mixture was cooled to 25-30° C. and non-polar impurities were removed by extraction with ethyl acetate. The crude almotriptan base (I) was obtained from the aqueous layer by neutralization, extraction with ethyl acetate and evaporation. The residue obtained was purified by converting it into an acid addition salt, either organic or mineral acid, to achieve the required impurity profile. Alternatively, the crude almotriptan base obtained as an oil was further easily purified by silica gel column chromatography (solvent system: dichloromethane: methanol: triethylamine, 9:1:0.5). The pale yellow oil was further converted into pharmaceutically acceptable salts.

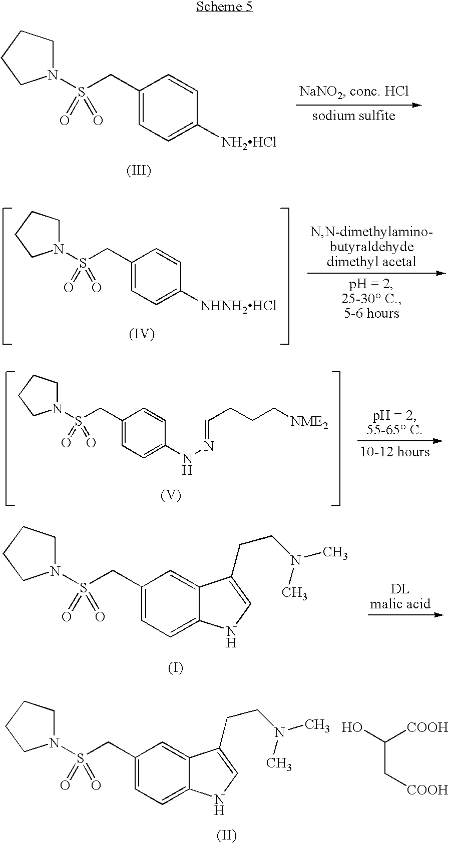

Alternatively, preparation of hydrazone (V) and its conversion into almotriptan was carried out using 1-(4-hydrazino-benzenemethanesulfonyl)pyrrolidine (IV) as starting material. A “one pot” synthesis of almotriptan by using 1-(4-hydrazino-benzenemethanesulfonyl)pyrrolidine hydrochloride (IV) is illustrated in Scheme 6. 1-(4-Hydrazino-benzenemethanesulfonyl)pyrrolidine hydrochloride (IV) was condensed with N,N-dimethylamino-butyraldehyde dimethyl acetal or another protected form, such as the diethyl acetal, to obtain hydrazone intermediate (V) followed by its cyclization to almotriptan.

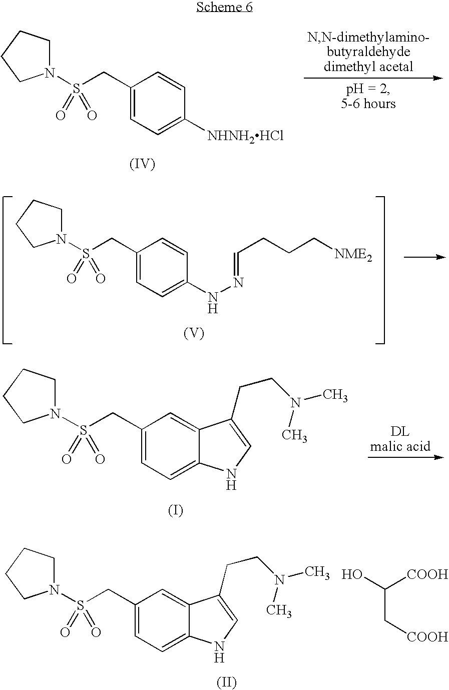

Alternatively the hydrazone intermediate (V) was isolated as an oil and cyclized to obtain almotriptan and pharmaceutically acceptable salts thereof (Schemes 7 and 8).

The preparation of almotriptan from isolated hydrazone intermediate (V) is illustrated in Scheme 7. Hydrazone intermediate (V), isolated as oil, was prepared from 1-(4-amino-benzenemethanesulfonyl)pyrrolidine hydrochloride (III). The reaction mixture was neutralized with sodium carbonate and separated hydrazone base (V) was extracted with ethyl acetate. The ethyl acetate layer was further washed with water to remove unwanted N,N-dimethylamino-butyraldehyde dimethyl acetal related impurities. The hydrazone (V) was obtained as oil by evaporation of ethyl acetate. The hydrazone base oil (V) was further subjected to cyclization to obtain almotriptan base by using a suitable cyclizing agent such as a mineral acid or Lewis acid and a suitable metal catalyst, e.g. hydrochloric acid, sulfuric acid, acetic acid, phosphoric acid, trifluoroacetic acid or boron trifluoride, and palladium (II) acetate. Crude almotriptan base (I) was obtained by usual aqueous work-up procedures comprising the steps of pH adjustment, extraction with ethyl acetate and evaporation of ethyl acetate. The crude almotriptan base oil was easily purified by silica gel column chromatography. The pale yellow oil was further converted into pharmaceutically acceptable salts.

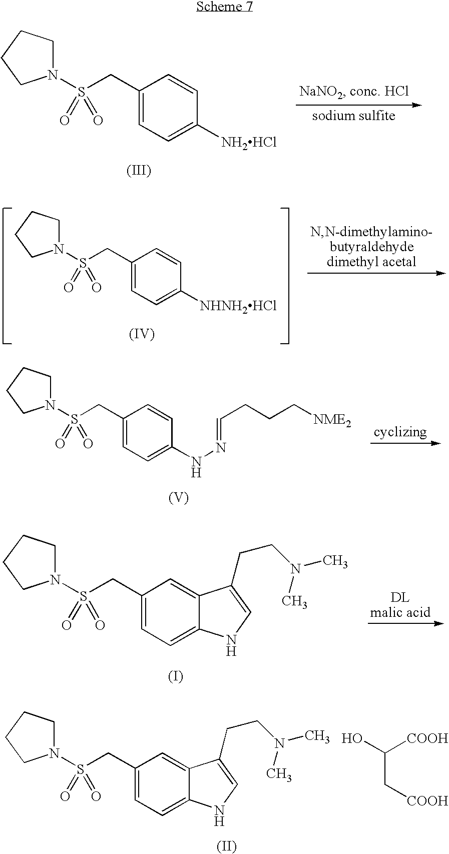

The preparation of almotriptan from isolated hydrazone intermediate (V) is also illustrated in Scheme 8. Hydrazone (V) was prepared from 1-(4-hydrazino-benzenemethanesulfonyl)pyrrolidine hydrochloride (IV) by following the process described in Scheme 6. The further steps (hydrazone isolation, cyclization and salt formation) were carried out as described in Scheme 7 to afford almotriptan.

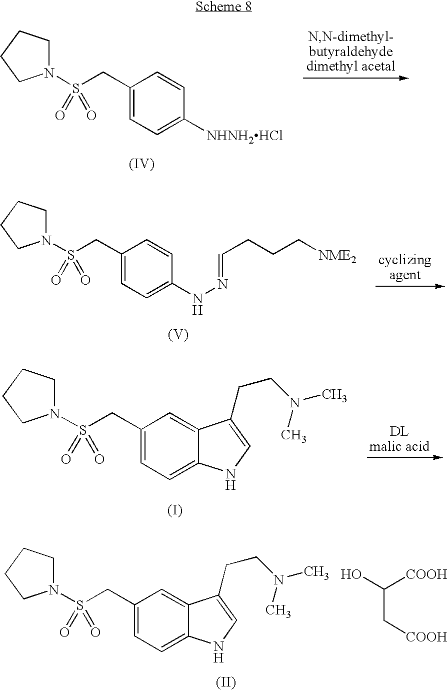

The following paragraphs enumerated consecutively from 1 through 54 provide for various aspects of the present invention. In one embodiment, the present invention provides:

1. A process for the preparation of almotriptan or a pharmaceutically acceptable salt thereof, comprising:
(a) condensation of 1-(4-hydrazino-benzenemethanesulfonyl)pyrrolidine, or a pharmaceutically acceptable salt thereof, with N,N-dimethylamino-butyraldehyde, or a protected form thereof, to form hydrazone intermediate (V), or a protected form thereof,

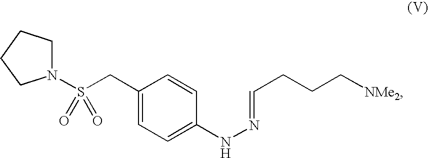

and
(b) cyclization of the hydrazone intermediate (V) to afford almotriptan.
2. A process according to paragraph 1, wherein the 1-(4-hydrazino-benzenemethanesulfonyl)pyrrolidine, or the pharmaceutically acceptable salt thereof, used in step (a) is prepared by diazotization of 1-(4-amino-benzenemethanesulfonyl)pyrrolidine, or a pharmaceutically acceptable salt thereof, followed by reduction.
3. A process according to paragraph 2, wherein the reduction is carried out by using stannous chloride, sodium dithionite or sodium sulfite.
4. A process according to paragraph 3, wherein the reduction is carried out by using sodium sulfite.
5. A process according to any preceding paragraph, which is a “one pot” process.
6. A process according to any preceding paragraph, wherein the 1-(4-hydrazino-benzenemethanesulfonyl)pyrrolidine, or the pharmaceutically acceptable salt thereof, is used in situ without isolation.
7. A process according to any of paragraphs 1 to 5, wherein the 1-(4-hydrazino-benzenemethanesulfonyl)pyrrolidine, or the pharmaceutically acceptable salt thereof, is isolated.
8. A process according to any preceding paragraph, wherein the hydrazone intermediate (V), or the protected form thereof, is used in situ without isolation.
9. A process according to any of paragraphs 1 to 7, wherein the hydrazone intermediate (V), or the protected form thereof, is isolated.
10. A process according to any preceding paragraph, wherein the pharmaceutically acceptable salt of the 1-(4-amino-benzenemethanesulfonyl)pyrrolidine or the 1-(4-hydrazino-benzenemethanesulfonyl)pyrrolidine used is the hydrochloride salt.
11. A process according to any preceding paragraph, wherein the condensation in step (a) is carried out at pH 0-3.
12. A process according to paragraph 11, wherein the condensation in step (a) is carried out at approximately pH 2.
13. A process according to any preceding paragraph, wherein the cyclization in step (b) is carried out at acidic pH.
14. A process according to paragraph 13, wherein the cyclization in step (b) is carried out at pH 0-3.
15. A process according to paragraph 14, wherein the cyclization in step (b) is carried out at approximately pH 2.
16. A process according to any preceding paragraph, wherein the cyclization in step (b) is carried out at 40-70° C.
17. A process according to paragraph 16, wherein the cyclization in step (b) is carried out at 55-65° C.
18. A process according to any preceding paragraph, wherein the cyclization in step (b) is carried out at high dilution.
19. A process according to paragraph 18, wherein the cyclization in step (b) is carried out at 10-100 volumes dilution.
20. A process according to paragraph 19, wherein the cyclization in step (b) is carried out at approximately 40 volumes dilution.
21. A process according to any preceding paragraph, wherein the cyclization in step (b) is carried out in the presence of one or more mineral acids or Lewis acids.
22. A process according to paragraph 21, wherein the mineral acid(s) or Lewis acid(s) is selected from hydrochloric acid, sulfuric acid, acetic acid, phosphoric acid, trifluoroacetic acid or boron trifluoride.
23. A process according to any preceding paragraph, wherein the cyclization in step (b) is carried out in the presence of a metal catalyst.
24. A process according to paragraph 23, wherein the metal catalyst is selected from palladium (II) acetate, palladium (II) chloride, Pd(P(C6H5)3)4, Pd2(dba)3, zinc chloride or ruthenium complexes.
25. A process according to paragraph 24, wherein the metal catalyst is palladium (II) acetate.
26. A process according to any preceding paragraph, further comprising the step of isolating the almotriptan formed by extraction using one or more organic solvents.
27. A process according to paragraph 26, wherein the organic solvent(s) is selected from methyl acetate, ethyl acetate, isopropyl acetate, dichloromethane, chloroform, diethyl ether, tertiary butyl methyl ether, diisopropyl ether or a mixture thereof.
28. A process according to any preceding paragraph, further comprising the step of isolating the almotriptan formed using an adsorbent and an elution system.
29. A process according to paragraph 28, wherein the adsorbent is selected from silica gel or a type of alumina.
30. A process according to paragraph 29, wherein the adsorbent is neutral alumina or basic alumina.
31. A process according to paragraph 29, wherein the adsorbent is silica gel.
32. A process according to any of paragraphs 28 to 31, wherein the elution system is selected from a mixture of a solvent and an organic base.
33. A process according to paragraph 32, wherein the solvent is an alcohol, acetate, chlorinated solvent or a mixture thereof.
34. A process according to paragraph 33, wherein the solvent is methanol, ethanol, isopropanol, methyl acetate, ethyl acetate, isopropyl acetate, butyl acetate, dichloromethane, chloroform, 1,2-dichloroethane or a mixture thereof.
35. A process according to any of paragraphs 32 to 34, wherein the organic amine is triethylamine, diethylamine, diisopropylamine, N-ethylisopropylamine, N,N-ethyldiisopropylamine, pyridine, pyrrolidone or a mixture thereof.
36. A process according to any preceding paragraph, wherein the N,N-dimethylamino-butyraldehyde is used in the form of an acetal.
37. A process according to paragraph 36, wherein the N,N-dimethylamino-butyraldehyde is used in the form of a diacetal.
38. A process according to paragraph 37, wherein the N,N-dimethylamino-butyraldehyde is used in the form of its dimethyl acetal or diethyl acetal.
39. A process according to paragraph 38, wherein the N,N-dimethylamino-butyraldehyde is used in the form of its dimethyl acetal.
40. A process according to any preceding paragraph, further comprising the step of preparing a pharmaceutically acceptable salt of almotriptan.
41. A process according to paragraph 40, for the preparation of almotriptan malate.
42. A process for the preparation of almotriptan or a pharmaceutically acceptable salt thereof, wherein the process utilizes hydrazone (V), or a protected form thereof:

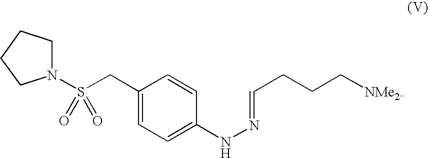

43. A process according to paragraph 42, for the preparation of almotriptan malate.
44. A process according to any preceding paragraph, wherein the almotriptan or the pharmaceutically acceptable salt thereof obtained has a chemical purity of 96% or more (as measured by HPLC).
45. A process according to any preceding paragraph, wherein the almotriptan or the pharmaceutically acceptable salt thereof is obtained in a yield of 20% or more from 1-(4-hydrazino-benzenemethanesulfonyl)pyrrolidine or a pharmaceutically acceptable salt thereof.
46. A process according to any preceding paragraph, wherein the almotriptan or the pharmaceutically acceptable salt thereof is obtained on an industrial scale.
47. Almotriptan or a pharmaceutically acceptable salt thereof, prepared by a process according to any preceding paragraph.
48. Almotriptan or a pharmaceutically acceptable salt thereof according to paragraph 47, wherein the pharmaceutically acceptable salt is almotriptan malate.
49. Almotriptan or a pharmaceutically acceptable salt thereof according to paragraph 47 or 48, for the treatment or prevention of migraine.
50. A pharmaceutical composition comprising almotriptan or a pharmaceutically acceptable salt thereof according to any of paragraphs 47 to 49.
51. Use of almotriptan or a pharmaceutically acceptable salt thereof according to any of paragraphs 47 to 49 in the preparation of a medicament for the treatment or prevention of migraine.
52. A method of treating or preventing migraine, comprising administering a therapeutically or prophylactically effective amount of almotriptan or a pharmaceutically acceptable salt thereof according to any of paragraphs 47 to 49 to a patient in need thereof.

53. A method according to paragraph 52, wherein the patient is a human.

54. A hydrazone represented by the formula (V), or a protected form thereof:

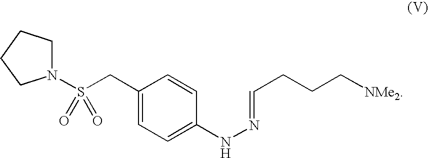

Further details of the invention, its objects and advantages are explained hereunder in greater detail in the following non-limiting examples.

### EXAMPLES

### Example 1

### One Pot Synthesis of Almotriptan from 1-(4-amino-benzenemethanesulfonyl)pyrrolidine hydrochloride (III)

1-(4-Amino-benzenemethanesulfonyl)pyrrolidine hydrochloride (III) (25 g) was charged in conc.

hydrochloric acid in 100 ml (4 vol.) water at 25-30° C. and the white suspension was stirred for 15 minutes before chilling to −5 to +5° C. A solution of sodium nitrite (10.7 g, 1.5 eq.) in 100 ml (4 vol.) water was added slowly over ½ hour at −5 to +5° C. to the white suspension. The resultant clear solution was stirred for 5 hours. Then the diazonium solution was transferred to an addition funnel and added slowly over 1 hour into a solution of sodium sulfite (78.5 g, 6 eq.) in 250 ml (10 vol.) of water at −5 to +5° C. The reaction mixture was stirred for 15 hours to achieve complete conversion of the diazonium compound to 1-(4-hydrazino-benzenemethanesulfonyl)pyrrolidine hydrochloride (IV). The solution of 1-(4-hydrazino-benzenemethanesulfonyl)pyrrolidine hydrochloride (IV) was further diluted with 500 ml (20 vol.) water, such that the total volume of the reaction mixture was in the range of 30-60 volumes. After dilution, N,N-dimethylamino-butyraldehyde dimethyl acetal 196 ml (10 eq.) was added to the hydrazine solution at 25-30° C. and the pH of the reaction mixture was checked (pH 9). The pH of the reaction mixture was adjusted to pH 2 by slow addition of 50% (v/v) HCl solution, about 12.5 ml (0.5 vol). The reaction mixture was stirred for 5-6 hours until complete conversion of 1-(4-hydrazino-benzenemethanesulfonyl)pyrrolidine hydrochloride (IV) to hydrazone (V) (by TLC) was achieved. The hydrazone (V) formed was cyclized to almotriptan base by heating the reaction mixture at 55-65° C. for 10-12 hours while maintaining the pH of the reaction mixture at pH 2. Then the reaction mixture was cooled to 25-30° C. and extracted with ethyl acetate 250 ml (10 vol.). The separated aqueous layer was neutralized with sodium carbonate (pH 8-9). The aqueous layer was extracted twice with ethyl acetate 500 ml (20 vol.). The ethyl acetate layer thus obtained was further washed twice with water. Almotriptan crude base was obtained as oil by removal of the ethyl acetate at reduced pressure. The crude almotriptan base was further purified by converting it into a suitable acid addition salt to obtain high quality almotriptan base. Alternatively, the almotriptan crude base was further purified by silica gel column chromatography by using a mixture of solvents (dichloromethane: methanol:triethylamine 9:1:0.5, or ethyl acetate: methanol:triethylamine 9:1:0.5).

Yield: 35% (w/w)

NMR data: ¹H NMR (300 MHz, CDCl₃) δ 1.76 (m, 4H), 2.35 (s, 6H), 2.63 (t, 2H), 2.93 (t, 2H), 3.14 (m, 4H), 4.37 (s, 2H), 6.99 (s, 2H), 7.19 (d, 1H), 7.27 (d, 1H), 7.56 (s, 1H), 8.60 (s, 1H).

Mass spectrum: 336.6 (M+1)

Purity: >99.85% (as measured by HPLC)

### Example 2

### One Pot Synthesis of Almotriptan from 1-(4-hydrazino-benzenemethanesulfonyl)pyrrolidine hydrochloride (IV)

1-(4-Hydrazino-benzenemethanesulfonyl)pyrrolidine hydrochloride (IV) (25 g) was added to water (1.25 L, 50 vol.) under stirring at 25-30° C. To the stirred suspension, N,N-dimethylamino-butyraldehyde dimethyl acetal (196 ml, 10 eq.) was added at 25-30° C. and the pH of the reaction mixture was checked (pH=9). The pH of the reaction mixture was adjusted to pH 2 by slow addition of 50% (v/v) HCl solution. The reaction mixture was stirred for 5-6 hours at pH 2 to achieve complete conversion of 1-(4-hydrazino-benzenemethanesulfonyl)pyrrolidine hydrochloride (IV) to hydrazone (V) (by TLC). Further cyclization of hydrazone (V) to almotriptan base (I), and isolation and purification of almotriptan base was carried out as in the experimental procedure described in example 1. Almotriptan crude base was further purified by converting it into a suitable acid addition salt to obtain high quality almotriptan base. Alternatively, almotriptan crude base was further purified by silica gel column chromatography by using a mixture of solvents (dichloromethane: methanol:triethylamine 9:1:0.5, or ethyl acetate: methanol:triethylamine 9:1:0.5).

Yield: 25% (w/w)

Purity: >99.85% (as measured by HPLC)

### Example 3

### Almotriptan preparation from hydrazone (V) isolated from 1-(4-amino-benzenemethanesulfonyl)pyrrolidine hydrochloride (III)

Hydrazone formation from 1-(4-amino-benzenemethanesulfonyl)pyrrolidine hydrochloride (III) was carried out by following the experimental procedure described in example 1. After confirmation of the hydrazone formation, the reaction mixture was basified with sodium carbonate solution to pH 8-9. The hydrazone was extracted twice with 125 ml (5 vol.) ethyl acetate and the ethyl acetate layer was further washed twice with water 125 ml (5 vol.). The hydrazone was isolated as oil by distillation of the ethyl acetate on a rotary evaporator at 45-50° C. at 50-100 mbar.

NMR data of hydrazone intermediate (V): ¹H NMR (300 MHz, CDCl₃) δ 1.4 (m, 2H), 1.80 (m, 6H), 2.35 (s, 6H), 2.52 (t, 2H), 3.25 (m, 4H), 4.25 (s, 2H), 6.60 (t, 1H), 6.90 (d, 2H), 7.27 (d, 2H), 9.80 (s, 1H).

Mass spectrum: 353 (M+1)

Further cyclization of hydrazone base oil (V) to almotriptan base (I), and isolation and purification of almotriptan base was carried out as in the experimental procedure described in example 1. Almotriptan crude base was further purified by converting it into a suitable acid addition salt to obtain high quality almotriptan base. Alternatively, almotriptan crude base was further purified by silica gel column chromatography by using a mixture of solvents (dichloromethane: methanol:triethylamine 9:1:0.5, or ethyl acetate: methanol:triethylamine 9:1:0.5).

Yield: 30% (w/w)

Purity: >99.85% (as measured by HPLC)

### Example 4

### Almotriptan Preparation from Hydrazone (V) Isolated from 1-(4-hydrazino-benzenemethanesulfonyl)pyrrolidine hydrochloride (IV)

Hydrazone formation from 1-(4-hydrazino-benzenemethanesulfonyl)pyrrolidine hydrochloride (IV) was carried out by following the experimental procedure described in example 2. Further cyclization of hydrazone base oil (V) to almotriptan base (I), and isolation and purification of almotriptan base was carried out as per the experimental procedure described in example 3.

Yield: 35% (w/w)

Purity: >99.85% (as measured by HPLC)

### Example 5

### Preparation of Almotriptan Malate from Almotriptan Base

Almotriptan base (5.0 g) was dissolved in 50 ml ethanol. To the clear pale yellow solution, malic acid (2.4 g in 50 ml ethanol) was added at 25-30° C. and the reaction mixture was stirred for 5 hours. After 5 hours, an off-white colored solid was discarded and the product was filtered and washed with 25 ml ethanol. The product was dried in a vacuum oven at 55-65° C. at 50-100 mbar for 6 hours to constant weight.

Yield: 85-90% w/w

m.p.: 167-169° C.

1. A process for the preparation of almotriptan or a pharmaceutically acceptable salt thereof, comprising:
(a) condensation of 1-(4-hydrazino-benzenemethanesulfonyl)pyrrolidine, or a pharmaceutically acceptable salt thereof, with N,N-dimethylamino-butyraldehyde, or a protected form thereof, to form hydrazone intermediate (V), or a protected form thereof, 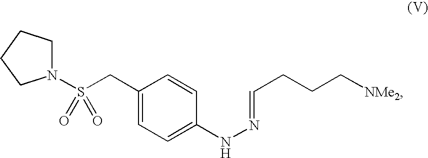
and
(b) cyclization of the hydrazone intermediate (V) to afford almotriptan.

2. A process according to claim 1, wherein the 1-(4-hydrazino-benzenemethanesulfonyl)pyrrolidine, or the pharmaceutically acceptable salt thereof, used in step (a) is prepared by diazotization of 1-(4-amino-benzenemethanesulfonyl)pyrrolidine, or a pharmaceutically acceptable salt thereof, followed by reduction.

3. A process according to claim 2, wherein the reduction is carried out by using stannous chloride, sodium dithionite or sodium sulfite.

4. A process according to claim 3, wherein the reduction is carried out by using sodium sulfite.

5. A process according to any preceding claim, which is a “one pot” process.

6. A process according to any preceding claim, wherein the 1-(4-hydrazino-benzenemethanesulfonyl)pyrrolidine, or the pharmaceutically acceptable salt thereof, is used in situ without isolation.

7. A process according to any of claims 1 to 5, wherein the 1-(4-hydrazino-benzenemethanesulfonyl)pyrrolidine, or the pharmaceutically acceptable salt thereof, is isolated.

8. A process according to any preceding claim, wherein the hydrazone intermediate (V), or the protected form thereof, is used in situ without isolation.

9. A process according to any of claims 1 to 7, wherein the hydrazone intermediate (V), or the protected form thereof, is isolated.

10. A process according to any preceding claim, wherein the pharmaceutically acceptable salt of the 1-(4-amino-benzenemethanesulfonyl)pyrrolidine or the 1-(4-hydrazino-benzenemethanesulfonyl)pyrrolidine used is the hydrochloride salt.

11. A process according to any preceding claim, wherein the condensation in step (a) is carried out at pH 0-3.

12. A process according to claim 11, wherein the condensation in step (a) is carried out at approximately pH 2.

13. A process according to any preceding claim, wherein the cyclization in step (b) is carried out at acidic pH.

14. A process according to claim 13, wherein the cyclization in step (b) is carried out at pH 0-3.

15. A process according to claim 14, wherein the cyclization in step (b) is carried out at approximately pH 2.

16. A process according to any preceding claim, wherein the cyclization in step (b) is carried out at 40-70° C.

17. A process according to claim 16, wherein the cyclization in step (b) is carried out at 55-65° C.

18. A process according to any preceding claim, wherein the cyclization in step (b) is carried out at high dilution.

19. A process according to claim 18, wherein the cyclization in step (b) is carried out at 10-100 volumes dilution.

20. A process according to claim 19, wherein the cyclization in step (b) is carried out at approximately 40 volumes dilution.

21. A process according to any preceding claim, wherein the cyclization in step (b) is carried out in the presence of one or more mineral acids or Lewis acids.

22. A process according to claim 21, wherein the mineral acid(s) or Lewis acid(s) is selected from hydrochloric acid, sulfuric acid, acetic acid, phosphoric acid, trifluoroacetic acid or boron trifluoride.

23. A process according to any preceding claim, wherein the cyclization in step (b) is carried out in the presence of a metal catalyst.

24. A process according to claim 23, wherein the metal catalyst is selected from palladium (II) acetate, palladium (II) chloride, Pd(P(C₆H₅)₃)₄, Pd₂(dba)₃, zinc chloride or ruthenium complexes.

25. A process according to claim 24, wherein the metal catalyst is palladium (II) acetate.

26. A process according to any preceding claim, further comprising the step of isolating the almotriptan formed by extraction using one or more organic solvents.

27. A process according to claim 26, wherein the organic solvent(s) is selected from methyl acetate, ethyl acetate, isopropyl acetate, dichloromethane, chloroform, diethyl ether, tertiary butyl methyl ether, diisopropyl ether or a mixture thereof.

28. A process according to any preceding claim, further comprising the step of isolating the almotriptan formed using an adsorbent and an elution system.

29. A process according to claim 28, wherein the adsorbent is selected from silica gel or a type of alumina.

30. A process according to claim 29, wherein the adsorbent is neutral alumina or basic alumina.

31. A process according to claim 29, wherein the adsorbent is silica gel.

32. A process according to any of claims 28 to 31, wherein the elution system is selected from a mixture of a solvent and an organic base.

33. A process according to claim 32, wherein the solvent is an alcohol, acetate, chlorinated solvent or a mixture thereof.

34. A process according to claim 33, wherein the solvent is methanol, ethanol, isopropanol, methyl acetate, ethyl acetate, isopropyl acetate, butyl acetate, dichloromethane, chloroform, 1,2-dichloroethane or a mixture thereof.

35. A process according to any of claims 32 to 34, wherein the organic amine is triethylamine, diethylamine, diisopropylamine, N-ethylisopropylamine, N,N-ethyldiisopropylamine, pyridine, pyrrolidone or a mixture thereof.

36. A process according to any preceding claim, wherein the N,N-dimethylamino-butyraldehyde is used in the form of an acetal.

37. A process according to claim 36, wherein the N,N-dimethylamino-butyraldehyde is used in the form of a diacetal.

38. A process according to claim 37, wherein the N,N-dimethylamino-butyraldehyde is used in the form of its dimethyl acetal or diethyl acetal.

39. A process according to claim 38, wherein the N,N-dimethylamino-butyraldehyde is used in the form of its dimethyl acetal.

40. A process according to any preceding claim, further comprising the step of preparing a pharmaceutically acceptable salt of almotriptan.

41. A process according to claim 40, for the preparation of almotriptan malate.

42. A process for the preparation of almotriptan or a pharmaceutically acceptable salt thereof, wherein the process utilizes hydrazone (V), or a protected form thereof:

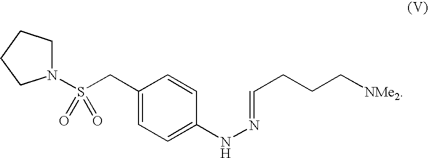

43. A process according to claim 42, for the preparation of almotriptan malate.

44. A process according to any preceding claim, wherein the almotriptan or the pharmaceutically acceptable salt thereof obtained has a chemical purity of 96% or more (as measured by HPLC).

45. A process according to any preceding claim, wherein the almotriptan or the pharmaceutically acceptable salt thereof is obtained in a yield of 20% or more from 1-(4-hydrazino-benzenemethanesulfonyl)pyrrolidine or a pharmaceutically acceptable salt thereof.

46. A process according to any preceding claim, wherein the almotriptan or the pharmaceutically acceptable salt thereof is obtained on an industrial scale.

47. Almotriptan or a pharmaceutically acceptable salt thereof, prepared by a process according to any preceding claim.

48. Almotriptan or a pharmaceutically acceptable salt thereof according to claim 47, wherein the pharmaceutically acceptable salt is almotriptan malate.

49. Almotriptan or a pharmaceutically acceptable salt thereof according to claim 47 or 48, for the treatment or prevention of migraine.

50. A pharmaceutical composition comprising almotriptan or a pharmaceutically acceptable salt thereof according to any of claims 47 to 49.

51. Use of almotriptan or a pharmaceutically acceptable salt thereof according to any of claims 47 to 49 in the preparation of a medicament for the treatment or prevention of migraine.

52. A method of treating or preventing migraine, comprising administering a therapeutically or prophylactically effective amount of almotriptan or a pharmaceutically acceptable salt thereof according to any of claims 47 to 49 to a patient in need thereof.

53. A method according to claim 52, wherein the patient is a human.

54. A hydrazone represented by the formula (V), or a protected form thereof: [chemical structure]
